(19) United States
(12) Patent Application Publication
(10) Pub. No.: US 2010/0249451 A1
(43) Pub. Date: Sep. 30, 2010

(54) PROCESS FOR THE PREPARATION OF SUBSTITUTED BENZILIC ACID FROM SUBSTITUTED BENZILS

(75) Inventors: Harshadas Mitaram MESHRAM, Hyderabad (IN); Palakuri Ramesh GOUD, Hyderabad (IN); Bandi Chennakesava REDDY, Hyderabad (IN); Busam Ramalinga Vara PRASAD, Hyderabad (IN); Mende SREENIVAS, Hyderabad (IN); Jhillu Singh YADAV, Hyderabad (IN)

Correspondence Address: LOWE HAUPTMAN HAM & BERNER, LLP, 1700 DIAGONAL ROAD, SUITE 300, ALEXANDRIA, VA, 22314, US

(73) Assignee: COUNCIL OF SCIENTIFIC & INDUSTRIAL RESEARCH, New Delhi (IN)

(21) Appl. No.: 12/726,967

(22) Filed: Mar. 18, 2010

(30) Foreign Application Priority Data
Mar. 27, 2009 (IN) 627/DEL/2009

(51) Int. Cl. C07C 51/16

(52) U.S. Cl. 562/418; 562/505; 562/527

(57) ABSTRACT

The classical process for the rearrangement of substituted benzil to benzilic acid is performed in the presence of sodium or potassium hydroxide as a base using ethanol-ether as a medium. The reaction requires reflux temperature for complete conversion. However, these bases containing metallic ions and generate metallic containing effluent waste which may require additional expenditure for treatment. Moreover, because of corrosive nature of base, and use of flammable solvent, the safety measures are needed during large scale production. Another method also reported for benzilic acid rearrangement at 380° C. which is practically not feasible. The present invention describes the use of quaternary ammonium hydroxides as a base for the rearrangement of the substituted benzils to benzilc acids. It also avoids the use of solvent and reaction can be carried out at relatively lower temperatures. Because of the solvent free reaction condition it reduces the mass/volume of reaction mixture.

### RELATED APPLICATIONS

The present application is based on, and claims priority from, India Application Number 627/DEL/2009 filed Mar. 27, 2009, the disclosure of which is hereby incorporated by reference herein in its entirety.

### FIELD OF INVENTION

The present invention relates to an improved process for the preparation of substituted benzilic acid from substituted benzils in the presence of quaternary ammonium hydroxides as a non-metallic base. Benzilic acid compounds are useful intermediates in the preparation of pharmaceuticals. Particularly, in the preparation of antidepressants, anticholinergics, antispasmodic drug and bronchodilator. Aluminium benzilic acid is an ingredient of the toner. Some of the substituted benzilic acid derivatives are used as acaricides and also in cosmetics as a skin conditioning agents.

### BACKGROUND OF THE INVENTION

The classical processes for the preparation of substituted benzilic acids are demonstrated by the rearrangement of substituted benzils in the presence of metallic base.

Reference may be made to U.S. Pat. No. 2,605,283 (1952). Wherein the alkoxy benzils are rearranged into benzilic acids in the presence of potassium hydroxide using ether as a solvent. This method has a drawback of using metal hydroxide as a base, which generates metal containing waste.

Another disadvantage of this process is the use of ether as a solvent which is highly flammable and low boiling volatile solvent. These facts may cause inconvenient on the large scale production.

Another reference may be made to U.S. Pat. No. 2,570,181 (1951). Where in the benzilic acid rearrangement carried out using potassium hydroxide in a mixture of ethanol-ether. This procedure requires longer reaction time and use of volatile solvents for reaction, makes this procedure inconvenient and expensive.

Yet reference may be made to The Ohio Journal of Science 53, (1), 31 Jan. 1953. Wherein p,p′-disubstituted benzils are converted into benzilic acids via rearrangement using potassium hydroxide in a mixture of ethanol-water. This method has drawback of using corrosive metallic base which generate metal ion containing waste. Another disadvantage is the use of expensive organic solvent, like ethanol. Moreover, the procedure is applicable to only p-substituted benzils.

Yet another reference may be made to J. Am. Chem. Soc. 75, 4769 (1953). Wherein the benzilic acid rearrangement performed using potassium hydroxide in water-ethanol medium. These particular procedures require chloroform to remove the impurity and benzene to extract the product. This may be inconvenient and expensive.

Yet another reference may be made to J. Chem. Soc. 1402 (1948). Wherein the rearrangement of benzil is carried out in presence of potassium tertiary butoxide. This process uses low boiling, volatile ether as a solvent media and reaction time also very long (44 hrs) to complete the reaction.

Yet another reference may be made to A Textbook of Practical Organic Chemistry A. I. Vogel, 5^{th} Edn; 1991. Longman Group Limited. London, p 808. Wherein the rearrangement is carried out using potassium hydroxide in a mixture of water & rectified spirit at 80-100° C. This particular procedure requires to keep the reaction mixture over night. Another disadvantage is that this method requires rectified spirit.

Yet another reference may be made to A Textbook of Practical Organic Chemistry A. I. Vogel, 5^{th} Edn; 1991. Longman Group Limited. London, p 808. Wherein rearrangement of benzil is performed using a mixture of sodium hydroxide and sodium or potassium bromate in water as a medium. Though this method has advantage of using water as a reaction medium, the use of metallic salt generates metal containing waste effluent. Moreover, this particular method has a drawback of forming benzhydrol as a side product.

Yet another reference may be made to Chemistry Letters (1990), 373. Wherein an improved procedure is described for the rearrangement of substituted benzils to benzilic acids using potassium hydroxide in solid state. The other metal hydroxides have also been reported for benzilic acid rearrangement. It is also stated that the rate of rearrangement in the solid state is more than in solution. This method has disadvantages of using solid potassium hydroxide which may be difficult to handle in large scale and it may also encounter the problem of proper mixing in solid state.

Yet another reference may be made to Green Chemistry (2005), 800. Wherein the rearrangement of benzils has been reported in high temperature water at 380° C. in the absence of catalyst. This particular procedure has disadvantages of using higher temperature and reaction leads to other side products.

All the earlier processes involve the use of metallic bases which are corrosive in nature and difficult to handle in large scale and create metal ion containing waste effluent. This necessitates, especially in the commercial scale, a high safety oriented and waste treatment expenditure which has negative influence on the economic viability of such process. Moreover, the use of a mixture alcohol-water as a reaction medium also expensive and additional step is require to remove alcohol. In addition to this, the reaction also requires higher temperature 80-100° C. Although practicable in laboratory, all these methods have following disadvantages on commercial scale.

1. Handling of corrosive potassium hydroxide is difficult.

2. Generates metal ion containing waste.

3. Additional waste treatment expenditure requires.

4. Safety measures are necessary.

5. Expensive alcohol is used as solvent.

6. Volatile and highly flammable solvents are used. Like ether is used.

7. Higher temperature requires for reaction

8. Additional step is required to remove alcohol.

9. Some of these methods have limitations to only p-substituted benzils.

10. Volume of reaction mass is more.

### OBJECTIVES OF THE INVENTION

The main object of the present invention is to provide an improved process for the preparation of substituted benzilic acid from substituted benzils in the presence of quaternary ammonium hydroxides as a non-metallic base.

Another object of the present invention is to develop a process without the use of corrosive metallic base.

Yet another object of the present invention is to avoid the generation of metal containing waste in large scale.

Still another object of the present invention is to avoid use of flammable and volatile solvent like ether or alcohol as a medium.

Yet another object of present invention is to reduce the volume of reaction mass by performing reaction in solvent free condition.

Yet another object of the present invention to carry out the reaction at lower temperature.

Yet another object of the present invention is to provide the general processes which may be applicable for all ortho, meta and para substituted benzils.

Still another object of the present invention is to avoid the possibility of contamination of metal ion in the pharmaceutical product.

### SUMMARY OF THE INVENTION

Accordingly, the present invention provides an improved process for the preparation of substituted benzilic acid from substituted benzils in the presence of quaternary ammonium hydroxides as a non-metallic base. The present invention provides a process for the rearrangement of substituted benzil to benzilic acid in high yield using quaternary ammonium hydroxides as a non-metallic base at moderate temperature wherein the reaction may be conducted in the absence of solvent or in aqueous medium. The main advantage of the invention is that it avoids the use of organic solvent and contamination of metal ions in pharmaceutical products.

The present invention provides a process for the production of substituted benzilic acid of formula 3, wherein R₁ and R₂ may be hydrogen, halogen, alkyl, alkoxy or nitro, by the rearrangement of substituted benzils of formula 1, wherein R₁ and R₂ may be hydrogen, alkyl, alkoxy, halogen or nitro, in the presence of quaternary ammonium hydroxides of formula 3 wherein the nitrogen is substituted with alkyl or aralkyl group.

In an embodiment of the present invention An improved process for the preparation of substituted benzilic acid of formula 3

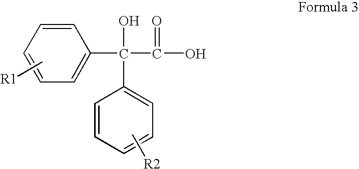

wherein R₁ and R₂ are selected from the group consisting of hydrogen, halogen (F, Cl, Br, I), alkyl (C₁-C₄), alkoxy (C₁-C₈) or nitro, wherein the said process comprising of:

a. stirring a mixture of substituted benzil of formula 1

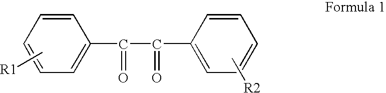

wherein R₁ and R₂ are selected from the group consisting of hydrogen, halogen, alkyl, alkoxy or nitro and quaternary ammonium hydroxide of formula 2

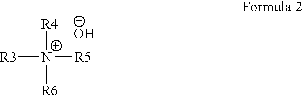

wherein R₃, R₄, R₅, R₆ may be alkyl (C₁-C₅), aralkyl or combination thereof at temperature ranging between 20° C. to 80° C., preferably 40° C. to 70° C. for a period ranging between 1 to 6 hrs, preferably 2 to 4 hrs;

b. diluting the reaction mixture as obtained in step (a) with water;

c. acidifying the reaction mixture as obtained in step (b) to pH ranging between 2 to 3.5 with dilute hydrochloric acid;

d. filtering and washing the solid as obtained in step (c) with water to obtained substituted benzilic acid, yield in the range of 85 to 95%.

In another embodiment of the present invention the substituent present on both the phenyl ring of the benzil are at ortho, meta or para position.

In another embodiment of the present invention the substituents on both the phenyl ring of the benzil are same or different.

In another embodiment of the present invention the substituents on both the phenyl ring of the benzil are at similar position or different.

In another embodiment of the present invention alkyl group in substituted benzil is unsubstituted straight chain hydrocarbon groups having 1 to 4 carbon atoms, preferably 1 to 3 carbon atoms.

In another embodiment of the present invention alkoxy group in substituted benzil is an aryl group bonded directly through an alkoxy group having carbon length C₁-C8, either straight chain or branch, preferably methoxy, ethoxy, propoxy.

In another embodiment of the present invention the quaternary ammonium hydroxide of formula 2 is nitrogen substituted with four alkyl or aralkyl group, wherein alkyl group having 1 to 5 carbons, preferably methyl or benzyl or combination thereof.

In another embodiment of the present invention quaternary ammonium hydroxide is used neat or as a 10 to 40% solution in water.

In another embodiment of the present invention the reaction is carried out in the absence of organic solvents or in aqueous condition, preferably in solvent free condition.

### DETAILED DESCRIPTION OF THE INVENTION

The present invention provides a process for the preparation of substituted benzilic acids by the rearrangement of substituted benzils in the presence of quaternary ammonium hydroxide as a non-metallic base. The aqueous solution of quaternary ammonium hydroxide having concentration of 10%, 20%, 30% 40% are suitable for quantitative conversion of benzil, preferably 30% gave the good results. Similarly, the reaction in solvent free condition also yield expected benzilic acid in high yield. The reaction can be performed either in water or in solvent free condition. The yield of the product is comparable in both the media. However, the solvent free media has advantage that it reduces the volume of reaction mass. The temperature ranging from 20° C. to 80° C. is suitable to complete the reaction, preferably at the range from 30° C. to 60° C. The time for reaction ranges from 1 to 6 hrs to complete the reaction, preferably 2 to 4 hrs.

FIG. 1 represents the rearrangement of substituted benzils to benzilic acids.

The duration of reaction depends on the substituent present on benzil. The reaction time is relatively shorter in case of halogen substituent as compared to other substituent. However, the procedure is suitable for the rearrangement of meta, para or ortho substituted benzil into benzilic acid. Similarly, the process is equally holds good for the rearrangement of symmetrical or unsymmetrical substituted benzil into benzilic acid.

The following examples are given to illustrate the process of the present invention and should not be construed to limit the scope of the present invention.

### Example 1

### Synthesis of Benzilic Acid

A mixture of benzil (0.1 mol) and Triton B (Benzyltrimethylammonium hydroxide) (0.2 mol) was heated at 40° C. for 2 hrs with stirring. The mixture was diluted with water and acidified with 10% hydrochloric acid up to pH −3. The solid filtered and wash with water to obtained benzilic acid in 92% yield.

### Example 2

### Synthesis of 4′-Chloro-Diphenyl Bezilic Acid

4′-Chloro benzil (0.1 mol) was taken in aqueous tetra methyl ammonium hydroxide (10 ml, 20% solution) and the suspension was heated at 50° C. for 3 hrs. The mixture was cooled and acidified with 1 N HCl up to pH 2.5. The solid filtered and wash with water to obtained 4′-Chloro-diphenyl benzilic acid in 95% yield.

### Example 3

### Synthesis of 4,4′-Difluorobenzilic Acid

A mixture of 4,4′-difluoro benzil (0.1 mol) and neat Triton B Benzyltrimethylammonium hydroxide (0.2 mol) was stirred at 20° C. for 6 hrs. The mixture was poured into cold water and acidified with 30% hydrochloric acid up to pH 2.7. The solid filtered and wash with water to obtained 4,4′-difluoro benzilic acid in 93% yield.

### Example 4

### Synthesis of 2,2′-Dichlorobenzilic Acid

Powdered 2,2′dichlorobenzil (0.1 mol) was added to a solution of tetra methyl ammonium hydroxide (10 ml, 25% solution) and the mixture heated at 50° C. for 6 hrs. After cooling, it was acidified with 50% hydrochloric acid up to pH −2.2. The solid filtered and wash with water to obtained 2,2′-dichloro benzilic acid in 85% yield.

### Example 5

### Synthesis of 4′-Methoxybenzilic Acid

A solution of Triton B (0.2 mol in 10 ml water) and 4′-methoxy benzil (0.1 mol) was heated at 60° C. for 5 hrs. The mixture was cooled and acidified with 30% hydrochloric acid up to pH 3. The solid filtered and wash with water to obtained 4′-methoxy benzilic acid in 94% yield.

### Example 6

### Synthesis of 3,3′-Dimethoxybenzilic Acid

Powdered 3,3′-dimethoxy benzil (0.1 mol) and Triton B (0.3 mol) were mixed and warmed at 50° C. for 5 hrs. The mixture was cooled to room temperature and acidified with 30% hydrochloric acid up to pH −3.1. The solid filtered and wash with water to obtained 3,3′-dimethoxy benzilic acid in 87% yield.

### Example 7

### Synthesis of 4,4′-Dinitrobenzilic Acid

A mixture of 4,4′-diniitrobenzil (0.1 mol) and Triton B (0.15 mol, neat) was heated at 80° C. for 3 hrs. The mixture cooled and acidified with 30% hydrochloric acid up to pH −2.5. The solid filtered and wash with water to obtained 4,4′-dinitro benzilic acid in 95% yield.

### Example 8

### Synthesis of α-Hydroxycycobutanecarboxylic Acid

A mixture of cyclopentane-1,2-dione (0.1 mol) and neat Triton B (0.2 mol) was stirred at 25° C. for 7 hrs. The mixture was poured into ice-cold water and acidified with 20% hydrochloric acid up to pH 2.6. The solid filtered and wash with cold water to obtained α-hydroxycycobutanecarboxylic acid in 89% yield.

### Example 9

### Synthesis of 2-Hydroxypropane-1,2,3-Tricarboxylic Acid

A solution of 3,4-dioxohexanedioic acid (0.1 mol) in Triton B (0.2 mol in 5 ml of water) was heated at 45° C. for 6 hrs. The mixture was cooled and acidified with 30% hydrochloric acid up to pH 2.9. The solid filtered and wash with cold water to obtained 2-hydroxypropane-1,2,3-tricarboxylic acid in 85% yield.

### ADVANTAGES

The present invention for the rearrangement of substituted benzils to benzilc acid has following distinct advantages.

1. Process is convenient and avoids use of metallic hydroxides.

2. Non-metallic base is used.

3. Alcohol or flammable volatile solvents like ether are not required for reaction.

4. The reaction is carried out at low temperature range.

5. Yields are high.

6. Applicable for symmetrical as well as unsymmetrical benzils.

7. Avoids the possibility of metal ion contamination in pharmaceutical products.

We claim:

1. An improved process for the preparation of substituted benzilic acid of formula 3 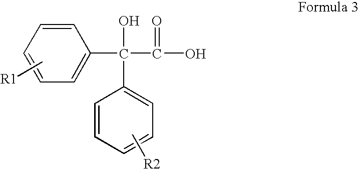
wherein R₁ and R₂ are selected from the group consisting of hydrogen, halogen(F, Cl, Br, I), alkyl (C₁-C₄), alkoxy (C₁-C₈) or nitro, wherein the said process comprising of:
a. stirring a mixture of substituted benzil of formula 1 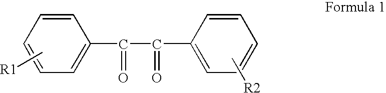
wherein R₁ and R₂ are selected from the group consisting of hydrogen, halogen, alkyl , alkoxy or nitro and quaternary ammonium hydroxide of formula 2 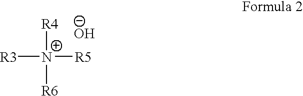
wherein R₃, R₄, R₅, R₆ may be alkyl (C₁-C₅), aralkyl or combination thereof at temperature ranging between 20° C. to 80° C., preferably 40° C. to 70° C. for a period ranging between 1 to 6 hrs, preferably 2 to 4 hrs;
b. diluting the reaction mixture as obtained in step (a) with water;
c. acidifying the reaction mixture as obtained in step (b) to pH ranging between 2 to 3.5 with dilute hydrochloric acid;
d. filtering and washing the solid as obtained in step (c) with water to obtained substituted benzilic acid, yield in the range of 85 to 95%.

2. A process as claimed in claim 1, wherein the substituent present on both the phenyl ring of the benzil are at ortho, meta or para position.

3. A process as claimed in claim 1, wherein the substituents on both the phenyl ring of the benzil are same or different.

4. A process as claimed in claim 1, wherein the substituents on both the phenyl ring of the benzil are at similar position or different.

5. A process as claimed in claim 1, wherein alkyl group in substituted benzil is unsubstituted straight chain hydrocarbon groups having 1 to 4 carbon atoms, preferably 1 to 3 carbon atoms.

6. A process as claimed in claim 1, wherein alkoxy group in substituted benzil is an aryl group bonded directly through an alkoxy group having carbon length C₁-C8, either straight chain or branch, preferably methoxy, ethoxy, propoxy.

7. A process as claimed in claim 1, wherein the quaternary ammonium hydroxide of formula 2 is nitrogen substituted with four alkyl or aralkyl group, wherein alkyl group having 1 to 5 carbons, preferably methyl or benzyl or combination thereof.

8. A process as claimed in claim 1, wherein quaternary ammonium hydroxide is used neat or as a 10 to 40% solution in water.

9. A process as claimed in claim 1, wherein the reaction is carried out in the absence of organic solvents or in aqueous condition, preferably in solvent free condition.

10. An improved process for the preparation of substituted benzilic acid from substituted benzils substantially as herein described with reference to the examples and drawings accompanying this specification.
